(19) United States
(12) Patent Application Publication
(10) Pub. No.: US 2013/0090392 A1
(43) Pub. Date: Apr. 11, 2013

(54) ONE STEP METHOD FOR THE PREPARATION OF WATER/OIL/WATER TYPE MULTIPLE EMULSION

(71) Applicant: Mamata Pradhan, Pune (IN)

(72) Inventor: Mamata Pradhan, Pune (IN)

(73) Assignee: Tata Consultancy Services Limited, Mumbai (IN)

(21) Appl. No.: 13/614,790

(22) Filed: Sep. 13, 2012

(30) Foreign Application Priority Data
Oct. 7, 2011 (IN) 2854/MUM/2011

(51) Int. Cl. A61K 9/113; A23D 7/00; A61K 8/06

(52) U.S. Cl. 514/785; 426/602

(57) ABSTRACT

The present invention relates to a process for preparing a multiple emulsion, particularly a water-in-oil-in-water emulsion in a single step using a single non ionic hydrophilic emulsifier that stabilizes the multiple emulsion for a period of one month. Further, the process uses low levels of a single emulsifier that makes the multiple emulsion find its application in any cosmetic, pharmaceutical and food industry catering to the specificity of the quantity of the emulsifier permissible within a particular industry and also acknowledging the quality of the final product required to be obtained for subsequent application and use thereof.

This application claims benefit of Serial No. 2854/MUM/2011, filed 7 Oct. 2011 in India and which application is incorporated herein by reference. To the extent appropriate, a claim of priority is made to the above disclosed application.

### FIELD OF THE INVENTION

The present invention relates generally to process for preparation of stable multiple emulsions and more particularly refers to a one step process for the preparation of stable water/oil/water (W/O/W) type multiple emulsion containing low levels of emulsifier.

### BACKGROUND OF THE INVENTION

Multiple emulsions are complex, heterogeneous systems in which both water-in-oil (W/O) and oil-in-water emulsions (O/W) coexist. Typically, a two-step emulsification process is used to make double emulsions (DEs), whereby the primary emulsion (W/O) is made using a lipophillic surfactant with low HLB value (typically <8). In the second step, the primary emulsion is gently emulsified with an external phase that contains a hydrophilic surfactant with high HLB value (typically >12), to produce water-in-oil-in-water (W/O/W) emulsion.

There are several works in prior art literature, where the double emulsion formation by one step method was observed but were found to be unstable. Stable double emulsions were produced by one step emulsification method with addition of both hydrophilic and lipophillic emulsifier at a single step. Most of these methods require many components and high amount of emulsifiers to form double emulsion via one step emulsification method.

These emulsions find their application for various purposes such as the production of cosmetics, pharmaceuticals and food products. Use of the emulsifiers in large quantities is undesirable as many consumers perceive these materials as potentially harsh or irritating to the skin, in case of cosmetics. The necessary addition of these emulsifiers not only adds cost to the final product, but also has an effect on the quality of such product, by affecting the way the emulsion breaks, as well as how it feels on the skin.

Similarly excessive use of emulsifiers in food products adversely affects the quality and texture of food. Further, on using the large amount of emulsifier, the multiple phase emulsion state gets destabilized and the emulsification rate is reduced by the effect of heat in the final stage, which is a serious demerit.

Though the addition of excessive amount of emulsifier solubilizes the primary emulsifier at the first stance, wherein the emulsifier is typically introduced in re-emulsification step, due to this re-emulsification of water in oil emulsion in water, it gets destabilized as the osmotic pressure differences between the internal and external water phases are often created.

In the light of foregoing limitations, there seems a long-felt, but unresolved need for a method for preparing a stable and cost effective multiple emulsion that can be suitably employed in any cosmetic, pharmaceutical and food industry catering to the specificity of the quantity of the emulsifier permissible within a particular industry and also acknowledging the quality of the final product required to be obtained for subsequent application and use thereof.

### OBJECTS OF THE INVENTION

It is a major object of the present invention to provide a one step method of preparing the W/O/W type multiple emulsion containing low levels of emulsifier.

It is an object of the present invention to provide a W/O/W type multiple emulsion of prolonged stability.

Another object of the present invention is to provide a stable W/O/W type multiple emulsions prepared using a single emulsifier.

It is an object of the invention to use low quantity of emulsifier in preparation of multiple emulsions thereby making it suitable for producing low fat edible emulsions used in food industry.

One of the other objects of the present invention is to prepare a multiple emulsion that can find a direct application in food industry without adversely affecting the quality and texture of food products.

Yet another object of the present invention is to produce multiple emulsions facilitating controlled release of water soluble species in personal care, cosmetics and pharmaceuticals.

In accordance with the aspect of the invention, the multiple emulsions produced have the broadest possible diversity of use, which does not have any disadvantage of prior art of excessive use of emulsifiers.

In another aspect of the present invention, cost effective multiple emulsions are produced that can be directly employed in preparing cosmetic, food or pharmaceutical formulations thereby reducing the overall cost of the final product.

### SUMMARY OF THE INVENTION

Briefly described and according to one aspect, the present invention provides a one step method of producing a stable multiple emulsion, particularly of water-in-oil-in-water type by using a single non ionic hydrophilic emulsifier. The extent of globule formation is however determined by the weight ratio of emulsifier to the oil phase and control of interfacial charging effect along with the ph of the aqueous phase.

According to another aspect of the invention a stable water-in-oil-in-water (W/O/W) type multiple emulsion comprising a principal aqueous phase of desired basicity and a principal oil phase of a particular concentration and the multiple emulsion containing a single non ionic hydrophilic emulsifier of a concentration less than or equal to its critical micelle concentration, is provided.

In yet another aspect of the invention, a process for the preparation of water-in-oil-in-water (W/O/W) type multiple emulsion is disclosed, the said process is characterized in using essentially a single emulsifier, wherein the process comprises of: adding an oil phase to an aqueous phase, the aqueous phase is emulsified with a non ionic hydrophilic emulsifier of a concentration less than or equal to its critical micelle concentration; and agitating the obtained emulsion while maintaining the basicity of aqueous phase at a given oil concentration.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

FIG. 1 is a representation of stages in formulation of W/O/W types multiple emulsion as determined in accordance with working example of the present invention.

### DETAILED DESCRIPTION OF THE INVENTION

Before the present method and composition are described, it is to be understood that this invention is not limited to the particular methodologies and ranges described, as these may vary within the specification indicated. It is also to be understood that the terminology used in the description is for the purpose of describing the particular versions or embodiments only, and is not intended to limit the scope of the present invention, which will be limited only by the appended claims. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. The disclosed embodiments are merely exemplary methods of the invention, which may be embodied in various forms.

The standard procedures practiced for the preparation of stable water-in-oil-in-water types multiple emulsions require many components and high amount of emulsifiers via one step emulsification method. In the present case, the water components can be any of the standard components that are ordinarily used for this purpose. The water phase, in the present invention, contributes to the volume of the inner droplet and the external continuous phase. The external continuous phase can be any suitable aqueous base depending upon the application to which the final emulsion is put to use. For example, in order to make the emulsion suitable for cosmetic industry, the aqueous phase can be any cosmetically acceptable base like de-ionized water or floral water.

Further, water in combination with certain proteins, gums, starch, salts, acid, colorants, seasoning or any medicine; or without such combination constitutes the aqueous phase of the invention.

The oil phase contributing to the volume of the outer droplet can be any cosmetically and pharmaceutically acceptable oil depending on the purpose for which it is intended. Preferably oil phase used in order to enhance multiple globule formation for the purposes of the present invention are the ones of higher molecular weight like n-alkanes. Although oils of lower molecular weight can also be used, and mixtures of high molecular weight oils and lower molecular weight n-alkanes is also contemplated, it is particularly preferred that the oil phase is composed of higher molecular weight, particularly higher molecular weight n-alkanes.

To prepare a water-in-oil-in-water type multiple emulsion, a single hydrophilic emulsifier of higher HLB value, ranging from 16-20, is used to accomplish the preparation of final emulsion in one single step. The extent of globule formation is determined by the weight ratio of emulsifier to the oil phase rather than emulsifier concentrations. The extent of multiplicity formation was decreased, with increase in concentration of emulsifier with respect to the oil phase. Particularly, extent of formation was more with high amount of oils and at higher pH. These multiple globules were stable for more than a month at room temperature against any physical treatment performed on the emulsion formed thereof

The process deviates from the existing processes by way of producing water-in-oil-in-water type emulsion in a single step and using low amounts of single emulsifier to make it adaptable for use in cosmetic bases, pharmacological applications as well as the food industry. Two step emulsification methods are complex and introduce possible destabilization pathways. For e.g. solubilization of the primary emulsifier may take place as a result of the excess surfactant that is typically introduced in the re-emulsification step, and osmotic pressure differences between the internal and external water phases are often created.

The major concern that the emulsion of the present invention addresses is the quantity and concentration of emulsifiers that have been traditionally used to prepare cosmetic, pharmaceutical and food compositions. The large amounts of emulsifier used made them undesirable for use in cosmetics, as many users found these materials potentially harsh and irritating to skin. Further, the necessity addition of these not only adds cost to the final product, but also has an effect on the quality of the final product, by affecting the way the emulsion breaks, as well as how it feels on the skin.

Similarly in food products, using large quantities of emulsifier for preparing food formulations were found to adversely affect the texture and quality of the final product. The emulsion can be suitably employed in preparation of dense mayonnaises, dressings, dips, whip topping coffee whitener, imitation ice creams and drinks.

Therefore, according to the present invention, various creams and lotions having a fresh, good feeling, an extremely high spread-ability and excellent cleansing effects and nutritive effects can be obtained. When the present invention is employed in the production of medicines, a physiologically active substance may be encapsulated in the inner aqueous phase to obtain a controlled-release medicine.

In accordance with the preferred embodiments of the present invention, a stable water-in-oil-in-water type multiple emulsion was produced using a single non ionic hydrophilic emulsifier. The extent of multiple globule formation is governed by the weight ratio of emulsifier to the oil phase. The emulsifier preferred for stabilizing the emulsion is polyoxyethylene (20) sorbitan monolaurate, popularly known as Tween 20. Polysorbate 20's hydrophile- lipophile balance is 16.7. The high HLB value makes it water soluble and suitable for stabilizing the emulsion as it easily travels into the water phase. The concentration of emulsifier used for preparing the multiple emulsions is equal to or less than its critical micelle concentration. This favors multiple globule formation at a very low concentration of emulsifier i.e. 0.1% based on the oil phase used.

The formation of multiplicity was reduced with increase in concentration ratio of emulsifier to oil phase. As known, the oil and water interface, in absence of an emulsifier is found to be negatively charged. In absence of emulsifier, a large portion of interfacial water molecules at the oil/water interface, have remarkably weak or negligible hydrogen bonding interactions and hence, the coordination number of water molecules is less at the interface relative to the bulk aqueous phase. The origin of negative charge is due to the specific orientation of OH- ions in water molecules towards oil molecules at the interface.

Phenomenologically, multiple emulsion formation originates from the interfacial charging effect during emulsification. Emulsification capacity of the emulsifier (at lower concentration) to produce single emulsion is being predominated by the interfacial charge. With increase in emulsifier (Tween 20) concentration possibly there is decrease in interfacial charge, because of more adsorption of surfactant molecules at the interface, which decreases (depletes) the area available for the adsorption of OH- ions. So less multiples were found at higher emulsifier concentration. The formation of multiplicity increased with increasing pH of the aqueous phase. The ph of the said aqueous phase is kept alkaline with a ph level maintained between 6-11. However, higher ph promoted multiple globule formation which is stable for a period of month.

Furthermore, destabilization pathways due to re-emulsification of water-in-oil emulsion in water, as observed in two step emulsification method is minimized by the process adopted in the present invention.

In one embodiment, for the process of preparing the W/O/W type multiple emulsion, oil in water emulsion containing an emulsifier of low concentration and high HLB value is prepared, which is agitated to allow formation of W/O/W type multiple globules at once. The basicity of aqueous phase is increased for multiple globule formation at a particular oil concentration and speed of agitation.

The agitation for the desired effect can be achieved using a homogenizer which makes the emulsion smooth and stable. The agitation with a homogenizer or any other way of mechanical agitation is effected preferably vigorously at a rate of 3000-8000 rpm. The type of homogenizer permissible to be used is not limited. A stirring device, like a Votator may be used for stirring and mixing the phases of the emulsion.

However, there is observed a separation of water layer at the bottom, soon after emulsification. The limitation is overcome to some extent and the water layer separation was reduced with use of fine oxide powders which are amphoteric / basic in nature without sacrificing multiplicity nature of the emulsion. The fine oxide powders are mainly amphoteric oxides, like, but not limited to magnesium oxide (MgO) or basic oxides like zinc oxides or other basic oxides. The oxide powder is added to the water (aqueous) phase to stabilize the oil droplets by Pickering method and prevent phase separation.

In an alternative embodiment of the present invention, oil soluble stabilizers are used to provide stabilization and prevent such phase separation.

### BEST MODE/EXAMPLE OF WORKING OF THE INVENTION

The embodiments were chosen and described in order to explain the principles of the methods and their practical application to enable others skilled in the art to utilize the methods and various embodiments and with various modifications as are suited to the particular use contemplated. Alternative embodiments will become apparent to those skilled in the art to which the present disclosure pertains without departing from their spirit and scope. Accordingly, the scope of the present inventions is defined by the appended claims rather than the foregoing description and the exemplary embodiments described therein.

A preparation of a multiple water-in-oil-in-water type multiple emulsion involves the use of Tween 20, a non ionic hydrophilic emulsifier to stabilize oil in water emulsion. If necessary, other emulsifiers may also be used suitably. This step further employs use of ultrasonication technique to obtain stable and well mixed emulsion. The technique generates alternating low pressure and high pressure waves in aqueous phase that causes strong shear forces needed for the disintegration of cells and better mixing of the two phases.

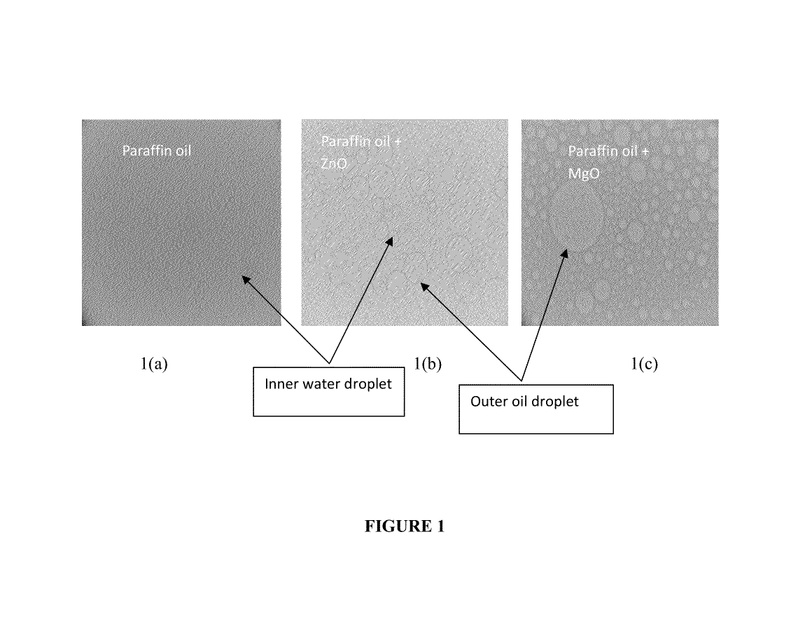

The concentration of oil phase used for the above preparation is 20%. The higher molecular weight oil is used as the dispersed medium, preferable n-alkanes like hexane, dodecane, Vaseline, lanolin, beeswax, paraffin oil or mineral oil or silicon oil; and not limited to animal or vegetable oil. As shown in FIG. 1(a) multiple globule formation of water-in-oil-in-water type is encouraged when the amount of emulsifier is around critical micelle concentration (CMC) or less than CMC of the emulsifier. The concentration is therefore maintained at 0.1 wt %, low enough to make it usable in diverse industries. Moreover, it is observed that with the increase in concentration of the emulsifier, the extent of formation was reduced.

Further, the extent of multiple globule formation is also dependent upon the time of emulsification which is preferably 2 minutes for the purposes of this invention and the extent of agitation. Almost 30% amplitude intensity is used for ultrasonication in order to achieve the desired strength during agitation.

With preparation of stable emulsifier, a water layer separation is also observed at the bottom soon after the emulsification process. The water layer separation layer can be reduced by the use of fine oxide powders. For example, the use of amphoteric oxide powder, say zinc oxide at a concentration of 0.075 wt % in water phase reduced the extent of water separation from emulsions, without sacrificing the extent of formation of multiple globules (as shown in FIG. 1(b)). With use of magnesium oxide (MgO) at the concentration of 0.075 wt %, though there was reduction in water phase separation but the extent of multiplicity formation was increased.

The reason for the formation of more multiples is change in pH of water or the aqueous phase from 6 to 11. With increase in pH, the interfacial charge was expected to be more, so more multiple formations was observed as shown in FIG. 1(c).

One of the other possible ways of stabilizing the above formed emulsion is by increasing the viscosity of the oil phase. Incorporated into this oil phase is an oil miscible thickener, for say, fat molecules that provides some level of thickening and assists in holding together different phases of the emulsion.

The embodiments were chosen and described in order to explain the principles of the systems and their practical application to enable others skilled in the art to utilize the systems and various embodiments and with various modifications as are suited to the particular use contemplated. Alternative embodiments will become apparent to those skilled in the art to which the present disclosure pertains without departing from their spirit and scope. Accordingly, the scope of the present inventions is defined by the appended claims rather than the foregoing description and the exemplary embodiments described therein.

We claim:

1) A process for the preparation of W/O/W type multiple emulsion, characterized in using essentially a single emulsifier, comprising of:
adding an oil phase to an aqueous phase, the aqueous phase emulsified with a non ionic hydrophilic emulsifier of a concentration less than or equal to its critical micelle concentration; and agitating the obtained emulsion while maintaining the basicity of aqueous phase at a given oil concentration.

2) The process of claim 1, further comprising the step of adding a fine oxide powder following the formation of multiple W/O/W type emulsion for reducing separation of aqueous layer upon emulsification.

3) The process of claim 1, wherein the single emulsifier is a non ionic hydrophilic emulsifier having an HLB of about 16 to 20.

4) The process of claim 1, wherein the single emulsifier is added to a concentration of equal to or less than the critical micelle concentration of the emulsifier to around 0.1% w/v.

5) The process of claim 1, wherein the oil phase used as a dispersed phase is added to a concentration of 20% to balance emulsifier to oil phase concentration for multiple globule formation.

6) The process of claim 1, wherein the basicity of aqueous phase is maintained by adjusting the ph of the aqueous phase in the range of 6-11, preferably at 11 for multiple globule formation.

7) The process of claim 1, wherein the oxide powder can be selected from a group comprising of zinc oxide or magnesium oxide or the combination thereof.

8) The process of claim 7, wherein the zinc oxide is preferred for reducing the water separation during the preparation of multiple emulsion.

9) The process of claim 1, wherein the extent of agitation is maintained in the range of 3000-8000 rpm to achieve multiple globule formation.

10) A stable W/O/W type double emulsion comprising a principal oil phase dispersed in an alkaline aqueous phase containing a single non ionic hydrophilic emulsifier of a concentration less than or equal to its critical micelle concentration.

11) The double emulsion of claim 10, wherein the emulsion remains stable for a period of not less than one month.

12) The double emulsion of claim 10, wherein the principal oil phase comprises oils of higher molecular weight, preferably selected from the group comprising of hexane, dodecane, paraffin oil, mineral oil and silicon oil.

13) The double emulsion of claim 10, wherein a non ionic hydrophilic emulsifier selected is polyoxyethylene sorbitan monolaurate.
